(19) United States
(12) Patent Application Publication
(10) Pub. No.: US 2021/0100909 A1
(43) Pub. Date: Apr. 8, 2021

(54) Polymeric Methylprednisolone Conjugates and Uses Thereof

(71) Applicants: United States Government As Represented By The Department of Veterans Affairs, Washington, DC (US); Board Of Regents Of The University Of Nebraska, Lincoln, NE (US)

(72) Inventors: Weiping Qin, Bronx, NY (US); William A. Bauman, Bronx, NY (US); Dong Wang, Omaha, NE (US)

(21) Appl. No.: 17/065,188

(22) Filed: Oct. 7, 2020

Related U.S. Application Data
(60) Provisional application No. 62/911,794, filed on Oct. 7, 2019.

(51) Int. Cl. A61K 47/58; A61K 45/06; A61P 25/00; A61P 29/00

(52) U.S. Cl. CPC A61K 47/58; A61P 29/00; A61P 25/00; A61K 45/06

(57) ABSTRACT

Disclosed herein are polymeric methylprednisolone conjugates and prodrugs as well as method of using the same. Disclosed herein are methods of treating a spinal cord injury in a subject comprising: systemically delivering a composition comprising a polymeric methylprednisolone conjugate to the subject. Disclosed herein are methods of enhancing neuroprotection in a subject after spinal cord injury comprising systemically delivering a composition comprising a polymeric methylprednisolone conjugate to the subject.

### CROSS-REFERENCE TO RELATED APPLICATIONS

This application claims the benefit of U.S. Provisional Patent Application No. 62/911,794, filed on Oct. 7, 2019, each of which is incorporated by reference herein in its entirety.

### STATEMENT REGARDING FEDERALLY SPONSORED RESEARCH

This invention was made with government support under grants 5I01RX02089-A2 and B2020-C awarded by Veterans Health Administration, Rehabilitation Research and Development Service and grant 1R01AR062680 awarded by the National Institute of Health. The government has certain rights in the invention.

### BACKGROUND

Spinal cord injury (SCI) causes loss of sensory, motor, and autonomic function. Over the past two decades, a number of promising therapies for SCI have been investigated, including surgical, non-pharmacological interventions (e.g., hypothermia), pharmacological agents (e.g., targeting myelin-associated inhibitors of regeneration), and cellular transplantation therapies (e.g., Schwann cells and human embryonic stem cells). Unfortunately, none of these therapies demonstrated sufficiently robust efficacy to be widely accepted by the clinical community.

To date, Methylprednisolone (MP) is the only FDA approved, clinically used agent for the treatment of acute SCI. The damaged axonal and neuronal cell membranes in the injured spinal cord undergo secondary damage when they depolarize, releasing neurotransmitters like glutamate in cytotoxic quantities, then accumulating intracellular calcium and undergoing lipid peroxidation. The relatively slow progression of the secondary injury response (several hours to days after the initial injury) provides a therapeutic window and forms the basis of the current clinical protocol for systemic administration of MP after SCI.

Most of the side effects of MP therapy are related to the high systemic dosage and associated toxicity, and the relatively modest neurological gains are due to inadequate and inefficient dosing to the injury site. Therefore, targeted MP delivery to the injury site will likely reduce systemic side effects, enhance its efficacy, and, ultimately, improve neurological outcome and clinical care. Recent advances on drug delivery technology by nanomedicines provide the ideal platform for a targeted delivery of the drug to diseased tissues/cells while maintaining an inactive form prior to its interaction with the molecular targets, and thereby preventing drugs from interacting with targets nonspecifically.

Disclosed herein is the use of polymeric methylprednisolone conjugates for systemic MP delivery that avoids toxic systemic affects.

### BRIEF SUMMARY

Disclosed are polymeric methylprednisolone conjugates.

Disclosed are compositions comprising any of the disclosed polymeric methylprednisolone conjugates.

Disclosed are methods of treating a spinal cord injury in a subject comprising systemically delivering a composition comprising a polymeric methylprednisolone conjugate to the subject.

Disclosed are methods of enhancing neuroprotection in a subject after spinal cord injury comprising systemically delivering a composition comprising a polymeric methylprednisolone conjugate to the subject.

Disclosed are methods of inhibiting oxidative stress in the spinal cord of a subject comprising systemically delivering a composition comprising a polymeric methylprednisolone conjugate to the subject.

Disclosed are methods of inhibiting lipid peroxidation in the spinal cord of a subject comprising systemically delivering a composition comprising a polymeric methylprednisolone conjugate to the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B show the occurrence of selective uptake of Nano-MP in the injured spinal cord. (A) Detection of Nano-MP in vivo at different time points post-injection. Male rats underwent complete T4 spinal cord transection or laminectomy only. Nano-MP-IRDye (1 mg/rat) was injected via tail veins immediately after SCI. Blood vessels in the rat ear were visible soon after injection, suggesting that Nano-MP was present in blood circulation. At days 2 and 3, blood vessels were not detectable in the rat ear, indicating that Nano-MP was metabolized and cleared from the circulation. (B) Nano-MP was detected in the injured spinal cord at day 3, but not that in control or laminectomy-only rats. Images were taken using a LI-COR imaging system (×4) and fluorescent microscopy (Bar=400 μm), respectively. N=10.

FIGS. 2A, 2B, 2C, 2D, and 2E show internalization of Nano-MP nanoparticles by microglia and astrocytes. Male rats underwent complete T4 spinal cord transection or laminectomy only. Nano-MP-Alexa 488 (2 mg/rat) was injected via tail veins immediately after SCI and detected in injured spinal cord at about 1 cm caudal to the lesion epicenter by confocal microscopy. (A) Selective uptake of Nano-MP in injured spinal cord. (B) Nano-MP was detected in non-neuron cells in the spinal cord. Motor neurons were stained by cresyl violet (Nissl Staining; magnification ×4). Accumulation of Nano-MP in CD11⁺ microglia (C) and GFAP⁺ astrocytes (D) (×20; N=10); white arrows show double-labeling in intact cells. Nano-MP-Alexa 488 [2C,D)] showed significantly higher fluorescent signal in spinal tissue at the injury site than CMCht/PAMAM-MP-FITC could achieve, as reported by Cerqueira et al. Small 2013, 9, No. 5, 738-749 [2E].

FIGS. 3A and 3B show polymeric methylprednisolone conjugate (nano-MP) attenuated SCI-induced oxidative stress. (A) Representative images showing the nitrotyrosine level in the spinal cord (longitudinally sectioned) and fluorescent intensity quantification in 4 experimental groups (n=4-5 per group); (B) Nano-MP protected spinal cord from SCI-induced MDA level increase. MDA level was measured using TBARS assay. *p<0.5, **p<0.01, ***p<0.001 by one way ANOVA.

FIGS. 4A and 4B show Nano-MP attenuated SCI-induced inflammation. Representative images showing the (A) TNF-α; (B) GFAP level in the spinal cord (longitudinally sectioned) and fluorescent intensity quantification in 4 experimental groups (n=4-5 per group). *p<0.5, **p<0.01, ***p<0.001 by one way ANOVA.

FIGS. 5A and 5B show polymeric methylprednisolone conjugate (nano-MP) protected spinal cord from SCI-induced apoptosis. (A) Representative images showing activated caspase-3 level in the spinal cord (longitudinally sectioned) and fluorescent intensity quantification in 4 experimental groups; (B) Apoptosis in the spinal cord was measured by TACS2 TdT-Fluor In Situ Apoptosis Detection Kit. Representative images and intensity quantification were shown in 4 experimental groups (n=4-5 per group). p<0.5, **p<0.01, ***p<0.001 by one way ANOVA; ##p<0.05 by t-test.

FIG. 6 shows the design of a Study. The time line for SCI surgery, administration of a bolus of MP or Nano-MP intravenously, and subsequent infusion of MP for 24 h is depicted. The times at which animals were euthanized for sample collection are also indicated.

FIGS. 7A-7E show changes in Body Weight, Food Intake and Glucose Metabolism. (A) Changes of body masses are shown. Body weights at sacrifice were normalized relative to body weight prior to spinal cord transection (pre-operative body weight). (B) Changes of food intake (g/day) are shown. (C) Changes of fasting glucose are shown. Levels of glucose metabolism-related mRNA Glut 4 (D) and G6pc (E) in gastrocnemius muscle are shown. Data are expressed as mean±SEM. n=10-12 animals per group. Significance of differences was determined using one-way analysis of variance with a Newman-Keuls test post hoc. *P<0.05, **P<0.01 and ***p<0.001 versus the indicated group; NS, no significant difference.

FIGS. 8A-8D show Nano-MP administration, compared to that of free MP, reduces adverse effects on muscle after acute SCI. (A) Weights for skeletal muscle normalized to body weight before anesthesia are shown. (B) Representative Hematoxylin eosin staining images of gastrocnemius muscle sections are shown. The arrows indicate necrotic fibers or fibers invaded by inflammatory cells. Bar=100 μm. (C) Quantification of fiber cross-sectional area (fCSA). (D) Fiber size distribution between experimental groups. Data are expressed as mean±SEM. n=10-12 animals per group. Significance of differences was determined by using one-way analysis of variance with a Newman-Keuls test post hoc. *P<0.05 and **P<0.01 versus the indicated group. NS: not significant.

FIGS. 9A-9H show effects of Nano-MP administration on Expression of Muscle Atrophy genes and proteins. Changes in atrophy-related mRNAs and/or proteins in gastrocnemius (A-D) and soleus muscle (E-H). Levels of mRNA and/or proteins for TNFα, MAFbx, MuRF1 and FOXO1 are shown as fold-change relative to Sham-SCI as indicated above each panel, and are normalized to levels present in the Sham-SCI group. Data are expressed as mean±SEM. n=10-12 animals per group. Significance of differences was determined by using one-way analysis of variance with a Newman-Keuls test post hoc. *P<0.05 and **P<0.01 versus the indicated group. NS: not significant.

FIGS. 10A, 10B, and 10C show Nano-MP administration, compared to that of free MP, reduces adverse effects on bone after acute SCI. (A) Areal bone mineral density (aBMD) measurements in each group are shown at the distal femur (a) and proximal tibia (b). (B) Representative micro-CT 3D images of trabecular microarchitecture are displayed. (C) Measurements are shown for: (a) trabecular bone volume per total tissue volume (BV/TV), (b) trabecular number (Tb.N, mm⁻¹), (c) trabecular thickness, (d) trabecular separation (Tb.Sp (mm)), (e) connectivity density (conn.D (mm⁻³)), and (f) structure model index (SMI). (g) Bone stiffness and (h) Failure load were estimated from micro-finite element analysis (μFEA). Data are expressed as mean±SEM. n=10-12 animals per group. Significance of differences was determined by using one-way analysis of variance with a Newman-Keuls test post hoc. *P<0.05 and **P<0.01 versus the indicated group. NS: not significant.

FIGS. 11A and 11B show effects of Nano-MP administration on bone gene expressions after SCI. Using RNA extracted from long bones, gene expressions related to bone resorption (4A) and formation (4B) were determined by real-time PCR analysis. (4A-a) TRAP, (4A-b) Calr TRAP, (4A-c) Intergrin β3, (4A-d) RANKL, (4A-e) OPG/RANKL ratio, (4B-a) osteocalcin, (4B-b) Runx2, and (4B-c) SOST. Gene expression was normalized by 18 s. Data are expressed as mean±SEM n=4-5 animals per group. Significance of differences was determined by using one-way analysis of variance with a Newman-Keuls test post hoc. *P<0.05, **P<0.01 and ***p<0.001 versus the indicated group. NS, no significant difference.

FIGS. 12A and 12B show changes of cortical architecture of the femur midshaft. (A) Representative micro-CT 3D-images of cortical microarchitecture are displayed. (B) Cortical bone volume over total tissue volume (BV/TV). Data are expressed as mean±SEM n=10-12 animals per group.

### DETAILED DESCRIPTION

The disclosed method and compositions may be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polymeric methylprednisolone conjugate ” includes a plurality of such polymeric methylprednisolone conjugate, reference to “the polymeric methylprednisolone conjugate ” is a reference to one or more polymeric methylprednisolone conjugate and equivalents thereof known to those skilled in the art, and so forth.

The following abbreviations are used throughout: methylprednisolone (MP); spinal cord injury (SCI); N2-hydroxypropyl methacrylamide (HPMA): IRDye® 800CW-MP-HPMA (MP-Nano-IRDye, MP-Nano-IRDye® 800CW, and Nano-MP-IRDye); Alexa Fluor 488-labeled HPMA-MP (MP-Nano-Alexa 488; Nano-MP-Alexa 488; and Nano-MP-Alexa); HPMA-methylprednisolone and HPMA-methylprednisolone are used interchangeably (MP-Nano, Nano-MP or HPMA-MP).

The term “prodrug” refers to an agent, which is converted into the active compound (the active parent drug) in vivo. Prodrugs are typically useful for facilitating the administration of the parent drug. The prodrug may also have improved solubility as compared with the parent drug in pharmaceutical compositions. Prodrugs are also often used to achieve a sustained release of the active compound in vivo.

By “treat” is meant to administer a polymeric methylprednisolone conjugate or composition disclosed herein to a subject, such as a human or other mammal (for example, an animal model), that has a spinal cord injury in order to prevent or delay a worsening of the effects of the disease or condition, or to partially or fully reverse the effects of the injury.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.”

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Polymeric Methylprednisolone Conjugates

Disclosed are polymeric methylprednisolone conjugates.

The phrase “polymeric methylprednisolone conjugate” as used herein refers to a conjugate formed between one or more molecules of methylprednisolone and a polymeric matrix in which the methylprednisolone is associated with the polymeric matrix by means of chemical (e.g., covalent, electrostatic) interactions.

In some aspects, a polymeric methylprednisolone conjugate can comprise two or more molecules of methylprednisolone. In some aspects, the weight percent of methylprednisolone in the polymeric methylprednisolone conjugate can be from around 0.01 mol % to 15 mol %. In some aspects, the weight percent of methylprednisolone in the polymeric methylprednisolone conjugate can be less than 10 mol %, but above 0.01 mol %.

As stated above, a polymeric methylprednisolone conjugate comprises one or more molecules of methylprednisolone and a polymeric matrix. The phrase “polymeric matrix” as used herein refers to a three-dimensional matrix which comprises, or substantially consists of (e.g., at least 50%, or at least 80%, or more), polymeric chains of one or more polymeric materials.

The terms “polymer” or “polymeric material” or “polymeric substance” are used interchangeably and as used herein refers to an organic substance composed of a plurality of repeating structural units (backbone units) covalently connected to one another. The term “polymer” as used herein encompasses organic and inorganic polymers and further encompasses one or more of a homopolymer, a copolymer or a mixture thereof (a blend). The term “homopolymer” as used herein describes a polymer that comprises one type of monomeric units and hence is composed of homogenic backbone units. The term “copolymer” as used herein describes a polymer that comprises more than one type of monomeric units and hence is composed of heterogenic backbone units. The heterogenic backbone units can differ from one another by the pendant groups thereof.

In some aspects, suitable polymers of the polymeric methylprednisolone conjugate can be biocompatible, non-immunogenic and non-toxic. In some aspects, the polymers can be water-soluble or water-insoluble. Water-soluble polymers can be used to stabilize drugs, as well as to solubilize otherwise insoluble compounds. In some aspects, the water-soluble polymer is a neutral water-soluble polymer. Examples of neutral water-soluble polymers are, but are not limited to, N2-hydroxypropyl methacrylamide (HPMA), methoxy HPMA, polyethylene glycol (including branched or block copolymers, which may be degradable via peptide sequences, ester or disulfide bonds, etc.), dextran, Guar Gum, cellulose and its derivatives, starch and its derivatives, polyoxazoline, polyvinyl alcohol (PVA), polyphosphazenes, zwitterionic polymers or copolymers of the following monomers: N-isopropylacrylamide, acrylamide, N,N-dimethylacrylamide, N-vinylpyrrolidone, 2-methacryloxyethyl glucoside, N-methylolacrylamide, and combinations thereof.

A polymeric matrix can be in a form of micelles, micro- or nano-spheres, micro- or nanoparticles, of entangled and/or cross-linked polymeric chains, and other forms.

A polymeric material (e.g., polymer from which the polymeric backbone of a polymeric conjugate as described herein is derived, or corresponds to, as discussed herein), can be or can comprise a biostable polymer, a biodegradable polymer or a combination thereof.

The term “biostable”, as used in this context, describes a substance (a compound or a polymer) that remains intact under physiological conditions (e.g., is not degraded in vivo).

The term “biodegradable” describes a substance which can decompose under physiological and/or environmental conditions into breakdown products. Such physiological and/or environmental conditions include, for example, hydrolysis (decomposition via hydrolytic cleavage), enzymatic catalysis (enzymatic degradation), and mechanical interactions. This term typically refers to substances that decompose under these conditions such that at least 50 weight percent of the substance decompose within a time period shorter than one year.

The term “biodegradable” also encompasses the term “bioresorbable”, which describes a substance that decomposes under physiological conditions to break down products that undergo bioresorption into the host-organism, namely, become metabolites of the biochemical systems of the host-organism.

In some aspects, the polymers of the polymeric methylprednisolone conjugates can further be charged polymers or non-charged polymers. Charged polymers can be cationic polymers, having positively charged groups and a positive net charge at a physiological pH; oranionic polymers, having negatively charged groups and a negative net charge at a physiological pH. Non-charged polymers can have positively charged and negatively charged group with a neutral net charge at physiological pH, or can be non-charged.

In some aspects, the polymer of the polymeric methylprednisolone conjugates can be a synthetic polymer or a naturally-occurring polymer. In some aspects, the polymer is a synthetic polymer.

In some aspects, the polymeric methylprednisolone conjugate further comprises a linker. In some aspects, the polymeric matrix can be conjugated to one or more molecules of methylprednisolone via a linker. In some aspects, the linker can be a cleavable linker. For example, a cleavable linker can have an ester, hydrazone, acetal, ether, thiol ether, or amide linker bond. In some aspects, the linker cleaves in an acidic environment thereby releasing the methylprednisolone from the polymeric compound. For example, the site of an injury can be an acidic environment and the Nano-MP can accumulate in the site of injury by diffusion thereby resulting in cleavage of the linker.

In some aspects, the polymeric methylprednisolone conjugate further comprises a label. In some aspects, the label can be a dye. Examples of labels are, but are not limited to, IRDye 800CW and its series, Alexa Fluor 488 and its series, radioisotopes (e.g., I¹²⁵, Cu⁶⁴) for SPECT and PET, and MR contrast agents (e.g. Ga³⁺).

In some aspects, the polymeric methylprednisolone conjugate is a prodrug. For example, the polymeric methylprednisolone conjugate prodrug can be an HPMA-methylprednisolone. In some aspects, the polymeric methylprednisolone conjugate prodrug can be HPMA-methylprednisolone wherein a hydrazone bond can be used to link the C3 carbonyl group of methylprednisolone to a HPMA copolymer. In some aspects, the hydrazone bond can be cleavable under an acidic environment. In some aspects, after systemic administration of a HPMA-methylprednisolone, the HPMA-methylprednisolone can passively target to the spinal cord injury site according to the ELVIS mechanism. In some aspects, the methylprednisolone conjugate prodrug can be selectively sequestered by inflammatory infiltrates and activated local cells (e.g., microglia and astrocytes) with high phagocytic activity. In some aspects, the methylprednisolone conjugate prodrug can be sorted into the subcellular lysosomal compartment, where the acidic pH (the pH value would further reduce under inflammatory conditions) can trigger the gradual cleavage of the hydrazone bond and the release of methylprednisolone (which is then the active parent drug).

### C. Compositions

Disclosed are compositions comprising any of the disclosed polymeric methylprednisolone conjugates. For example, disclosed compositions can comprise one or more polymeric methylprednisolone conjugates. In some aspects, the disclosed compositions can further comprise a pharmaceutically acceptable carrier.

### 1. Delivery of Compositions

In the methods described herein, delivery (or administration) of the compositions to a subject can be via a variety of mechanisms. As defined above, disclosed herein are compositions comprising any one or more of the polymeric methylprednisolone conjugates described herein and can also include a carrier such as a pharmaceutically acceptable carrier. For example, disclosed are pharmaceutical compositions, comprising the polymeric methylprednisolone conjugates disclosed herein, and a pharmaceutically acceptable carrier.

“Pharmaceutically acceptable” as used herein refers to material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polymeric methylprednisolone conjugates is not compromised. Pharmaceutical compositions can also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### D. Methods

Disclosed are methods of treating a spinal cord injury in a subject comprising systemically delivering any of the disclosed compositions comprising a polymeric methylprednisolone conjugate to the subject.

Also disclosed are methods of enhancing neuroprotection in a subject after spinal cord injury comprising systemically delivering any of the disclosed compositions comprising a polymeric methylprednisolone conjugate to the subject.

In some aspects of the disclosed methods, the spinal cord injury can be an acute spinal cord injury.

Disclosed are methods of inhibiting oxidative stress in the spinal cord of a subject comprising systemically delivering any of the disclosed compositions comprising a polymeric methylprednisolone conjugate to the subject.

Disclosed are methods of inhibiting lipid peroxidation in the spinal cord of a subject comprising systemically delivering any of the disclosed compositions comprising a polymeric methylprednisolone conjugate to the subject.

In some aspects of the disclosed methods, the composition can be administered intravenously. In some aspects of the disclosed methods, the composition can be administered intraperitoneally.

In some aspects of the disclosed methods, the polymeric methylprednisolone conjugate accumulates at the spinal cord injury site. In some aspects, the polymeric methylprednisolone conjugate is specific to astrocytes and microglia. For example, the polymeric methylprednisolone conjugate can specifically accumulate in CD11+ microglia and GFAP+ astrocytes.

In some aspects of the disclosed methods, inflammation in the subject is reduced in the spinal cord. In some aspects of the disclosed methods, injury-related cellular markers are reduced in the spinal cord of the subject. For example, tumor necrosis factor alpha (TNF-α) accumulates in the injured area within days of an acute spinal cord injury. Treatment of the spinal cord injury with one or more of the disclosed polymeric methylprednisolone conjugates can result in reduced levels of TNF-α within days of treatment.

In some aspects of the disclosed methods, motor neuron apoptosis is decreased in the spinal cord of the subject.

In some aspects, only a single dose of the composition is delivered to the subject. In some aspects, more than one dose can be delivered to the subject. The doses can occur hours, days, weeks, or months apart. In some aspects, the delivery can be a continuous delivery wherein the continuous delivery occurs for 1, 2, 3, 4, 5, 6, 7, 8, 9, 10, 11, 12, 13, 14, 15, 16, 17, 18, 19, 20, 21, 22, 23, or 24 hours. In some aspects, the delivery can be a continuous delivery wherein the continuous delivery occurs for 1, 2, 3, 4, 5, 6, or 7 days. In some aspects, the delivery can be a continuous delivery wherein the continuous delivery occurs for 1, 2, 3, or 4 weeks. In some aspects, the delivery can be a continuous delivery wherein the continuous delivery occurs for 1, 2, 3, 4, 5, 6, 7, 8, 9, 10, 11, or 12 months. In some aspects, the delivery can be a continuous delivery wherein the continuous delivery occurs for 1, 2, 3, 4, 5, 6, 7, 8, 9, 10 years.

In some aspects of the disclosed methods, the polymeric methylprednisolone conjugate can be delivered at a dose of 60 mg/kg. In some aspects of the disclosed methods, the polymeric methylprednisolone conjugate can be delivered at a dose of 1 mg/kg-1000 mg/kg.

In some aspects of the disclosed methods, glucose metabolism is not impaired in the subject. Methylprednisolone administered by itself can have adverse effects on fasting glucose levels and carbohydrate intolerance. However, in some aspects, the disclosed polymeric methylprednisolone conjugates does not have any negative effects on fasting glucose levels and lower carbohydrate intolerance is present.

In some aspects of the disclosed methods, muscle and bone mass are not reduced in the subject. Loss of muscle and bone mass is a negative side effect to many steroid therapies. Use of the disclosed polymeric methylprednisolone conjugates can prevent or reduce the levels of muscle and/or bone mass loss. In some aspects, the muscle loss corresponds to reduced body weight and therefore, the polymeric methylprednisolone conjugates can also prevent or reduce loss of body weight.

In some aspects of the disclosed methods, a pain management therapeutic or anti-inflammatory agent can further be administered to the subject. In some aspects, the pain management therapeutic or anti-inflammatory agent can be co-administered with one or more of the disclosed polymeric methylprednisolone conjugates. In some aspects, the polymeric methylprednisolone conjugates and pain management therapeutic or anti-inflammatory agent can be administered simultaneously. In some aspects, the polymeric methylprednisolone conjugates and pain management therapeutic or anti-inflammatory agent can be co-administered in a single formulation. In some aspects, the polymeric methylprednisolone conjugates and pain management therapeutic or anti-inflammatory agent can be administered in separate formulations. Thus, regardless of whether the polymeric methylprednisolone conjugates and pain management therapeutic or anti-inflammatory agent are formulated together in a single formulation or in separate formulations, they can still be administered simultaneously. Simultaneous administration can include administering the polymeric methylprednisolone conjugates and pain management therapeutic or anti-inflammatory agent at the exact same time, within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes of each other. In some aspects, the polymeric methylprednisolone conjugates and pain management therapeutic or anti-inflammatory agent are administered consecutively.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the disclosed polymeric methylprednisolone conjugates or compositions comprising any of the disclosed polymeric methylprednisolone conjugates.

### EXAMPLES

### A. Target-Delivery of Low Dose of Nanomedicine-Enabled Methylprednisolone Sufficiently Attenuates Secondary Injury to Spinal Cords in Rats after Acute Spinal Cord Injury

### 1. Introduction

Spinal cord injury (SCI) causes loss of sensory, motor, and autonomic function. Over the past two decades, a number of promising therapies for SCI have been investigated, including surgical, non-pharmacological interventions (e.g., hypothermia), pharmacological agents (e.g., targeting myelin-associated inhibitors of regeneration), and cellular transplantation therapies (e.g., Schwann cells and human embryonic stem cells). Unfortunately, none of these therapies demonstrated sufficiently robust efficacy to be widely accepted by the clinical community.

To date, methylprednisolone (MP) is the only FDA approved, clinically used agent for the treatment of acute SCI. The damaged axonal and neuronal cell membranes in the injured spinal cord undergo secondary damage when they depolarize, releasing neurotransmitters like glutamate in cytotoxic quantities, then accumulating intracellular calcium and undergoing lipid peroxidation. The relatively slow progression of the secondary injury response (several hours to days after the initial injury) provides a therapeutic window and forms the basis of the current clinical protocol for systemic administration of MP after SCI.

Beginning in the late 1970s, three large nation-wide studies have been conducted to investigate the clinical efficacy of MP to improve function when administered soon after an SCI. The first National Spinal Cord Injury Study (NASCIS I) trial enrolled acute SCI patients for one of two treatments: a bolus of 100 mg or 1000 mg IV MP daily for 10 days. Infection rates rose for both dose groups while MP dosage did not impact functional outcomes. In 1990, the NASCIS II trial reported its comparison of MP, naloxone and placebo and again showed no difference between the groups. However, a subgroup of patients identified on post hoc analysis that were treated within 8 hours of injury with MP (30 mg/kg bolus followed by 5.4 mg/kg over 23 h) had better motor scores compared to the placebo group. Based on this tenuous result, MP quickly became established as a standard of care. In 1997 and 1998, based on findings of the NASCIS III trial, it was recommended to initiate a 24 hour course of MP if treatment was started within 3 hours of injury, using the same NASCIS II protocol, and a 48 hour course if treatment was started within 3-8 hours of injury.

Although MP showed modest benefits in a secondary analysis of the NASCIS II trial and continues to be commonly used in the US, the use of systemic high-dose MP in acute SCI remains controversial because of the modest neuroprotective effects, which must be weighed against potential side effects, which include susceptibility to infection and wound complications, gastric bleeding, sepsis, diabetic complications, pneumonia and acute corticosteroid myopathy. Because of these concerns, MP is no longer considered as a standard of care for the treatment of acute SCI, which has been, and this is reflected in the Guidelines for the Management of Acute Cervical Spine and Spinal Cord Injuries, which lists it as a treatment option.

Most of the side effects of MP therapy are related to the high systemic dosage and associated toxicity, and the relatively modest neurological gains are due to inadequate and inefficient dosing to the injury site. Therefore, targeted MP delivery to the injury site will likely reduce systemic side effects, enhance its efficacy, and, ultimately, improve neurological outcome and clinical care. Recent advances on drug delivery technology by nanomedicine provide the ideal platform for a targeted delivery of the drug to diseased tissues/cells while maintaining an inactive form prior to its interaction with the molecular targets, and thereby preventing drugs from interacting with targets nonspecifically.

One such approach is the synthesis of polymer conjugates, in which drugs are covalently attached to the polymer carrier via a cleavable linker [e.g., N2-hydroxypropyl methacrylamide (HPMA), polyethylene glycol (PEG)] that can be degraded in specific biological microenvironments. In principle, in presence of inflammation, nanocarriers such as polymers or liposomes will preferentially extravasate through leaky vasculature and then be sequestered and retained by the cell infiltrates at the inflammation sites, resulting in the passive targeting of the nanomedicine to sites of inflammation (Quan, Purdue et al. 2010).

In this study, the use of nanomedicine (HPMA)-based, site-specific MP delivery onto the injured spinal cord in rats was investigated. Analysis was performed at 2 days and 7 days post injury and used to quantify the influence on secondary responses including oxidative stress, inflammation and apoptosis. Results were quantified and compared with laminectomy only animals (Sham), vehicle-treated SCI animals (SCI-veh) and systemically administrated MP (30 mg/kg, single dose) animals (SCI-MP).

### 2. Material and Methods

### i. Animals and Surgery

All animals were maintained on a 12:12-h light/dark cycle with lights on at 07:00 h in a temperature-controlled (20±2° C.) vivarium, and all procedures were approved by the JJP VA Medical Center IACUC. Spinal cord transection surgery was performed as previously described. In brief, 9-week-old Wistar rats (Charles River) were anesthetized by inhalation of isofluorane (3-5%) and hair was removed with a clipper. Skin over the back was cleaned with betadine and isopropyl alcohol. After making a midline incision the spinal cord at the site of transection (T4) was visualized by laminectomy, and the spinal cord was transected with microscissors. The space between transected ends of the spinal cord was filled with surgical sponge and the wound was closed in two layers with suture. Urine was manually expressed 3 times daily until automaticity develops, then as needed. Baytril was administered for the first 3 to 5 days postoperatively then as needed for sign of cloudy or bloody urine or wound infection. Sham-transected animals received a laminectomy-only surgery.

### ii. Nanoparticle Synthesis and Drug Administration

Injectable methylprednisolone (MP) solution (62.5 mg/ml) was obtained from Pfizer. MP was conjugated to the chain terminus of methoxy HPMA, an amphiphilic macromolecular prodrug. In parallel, IRDye® 800CW-MP-HPMA (MP-Nano-IRDye) and Alexa Fluor 488-labeled HPMA-MP (MP-Nano-Alexa 488) were also synthesized for optical imaging to visualize the bio-distribution of MP-Nano. Immediately after spinal cord transection, animals were administered an intravenous injection of freshly prepared MP-Nano (60 mg/kg, equivalent to 20% free MP dose at 30 mg/kg), MP (30 mg/kg) or vehicle (propylene glycol) by tail vein, followed by implantation of an Alzet pump (Durect Co, Cupertino, Calif., USA) which provided a 24-h infusion of MP at 5.4 mg/kg/hr (SCI-MP), or vehicle (propylene glycol) (SCI-veh and SCI-Nano-MP) into a subcutaneous pocket. These dosing of MP corresponds on an mg per kg basis to that prescribed by the Bracken protocol. Sham-transected animals underwent laminectomy and implantation of the same Alzet pumps, with vehicle (propylene glycol) infusion instead, as baseline controls.

### iii. Examination of the Bio-Distribution of MP-Nano

The animals (N=10) was anesthetized (inhalation of isofluorane) and imaged prior to surgeries and then daily post-surgery using an in vivo Imaging System (LI-COR, Lincoln, Nebr., USA) to evaluate the distribution of the MP-Nano daily for 3 days.

### iv. Tissue Preparation and Histological Analysis of Spinal Cord

At 2 and 7 days after injury, animals (sham, SCI-veh, SCI-MP, SCI-Nano-MP, n=5 animals for each condition and time point) were perfused transcardially with 4% paraformaldehyde. The spinal cords were removed and post fixed overnight in the same fixative. Longitudinal 10 μm thick tissue sections were cryosectioned. Serial sections were blocked for 1 hour at room temperature in blocking buffer (1×PBS/5% Goat serum/0.3% Triton X-100), followed by incubation of primary antibodies including: tumor necrosis factor alpha (TNF-α, Millipore); glial fibrillary acidic protein (GFAP, DAKO Cytomation) to identify astrocytes; Nitrotyrosine (Millipore) as an oxidative stress marker; μ-Calpain (Sigma); Bcl-2-associated X protein (Bax, Santa Cruz Biotechnologies) as a pro-apoptotic marker and activated Caspase3 (Cell Signaling) as an apoptotic marker. After washing and incubation with either Alexa Fluor 488 or Alexa Fluor 647-conjugated secondary antibodies (Cell signaling), slides were mounted and subjected to fluorescent microscopy. For animals that received MP-Nano-Alexa 488 injection, slides were either immunostained with GFAP followed by Alexa 647-conjugated secondary antibody (Cell signaling) or directly stained with cresyl violet (Sigma). Images were taken using the EVOS FL Auto system (Life Technologies). Fluorescence intensity quantifications were performed in the ImageJ software (NIH).

### v. Lipid Peroxidation

At two days post injury, sections of spinal cord centered around the site of lesion or laminectomy were dissected out and stored at −80° C. Spinal cords were then lysed in RIPA buffer and lipid peroxidation was measured using the TBARS Assay Kit (Cayman Chemical) following the manufacturer's instructions. Malondialdehyde (MDA) concentration was determined using the colorimetric method.

### vi. In Situ Apoptosis Detection

Apoptosis was measured using the TACS2 TdT-Fluor In Situ Apoptosis Detection Kit (Trevigen) following the manufacturer's instructions. In brief, longitudinal 10 μm thick spinal cord sections were incubated with Cytonin for 1 hour at room temperature and labeled with Labeling Reaction Mix for 1 hour at 37° C. After the reaction was stopped, samples were washed twice with PBS and incubated with Strep-Fluor solution for 20 minutes in the dark, washed twice with PBS again, mounted and subjected to fluorescent microscopy.

### vii. Statistics

Data are expressed as mean±SE; the number of independent samples (n) is provided in the legend of each figure. The statistical significance of differences among means was tested using one-way analysis of variance and Bonferroni's post hoc test to determine the significance of differences between individual pairs of means using a p value of 0.05 as the cutoff for significance. Statistical calculations were performed using Prism 4.0c (GraphPad Software, La Jolla, Calif., USA).

### 3. Results

### i. Selective Uptake of Nano-MP Nanoparticles in the Injured Spinal Cord

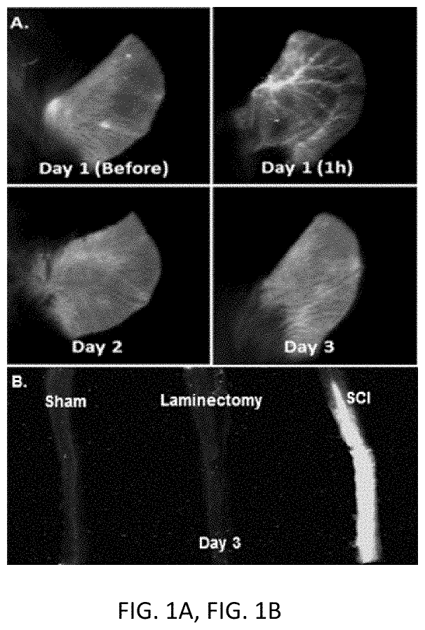

To visualize MP-Nano distribution in vivo, two fluorescently tagged derivatives were co-administered to permit examination of tissue levels by IR and conventional fluorescence imaging: IRDye® 800CW-labeled MP-Nano (1 mg/rat), and MP-Nano-Alexa 488 (2 mg/rat). The biodistribution of the MP-Nano was examined using a LI-COR Imaging System daily for 3 days. MP-Nano-IRDye® 800CW was observed 1 hour after injection and continued to be present for at least 3 days (FIG. 1A). Interestingly, only SCI animals, not control or laminectomy animals, showed MP-Nano-IRDye® 800CW distribution in the spinal cord (FIG. 1B), indicating that the nanoparticle-conjugated MP was selectively delivered to the injury site.

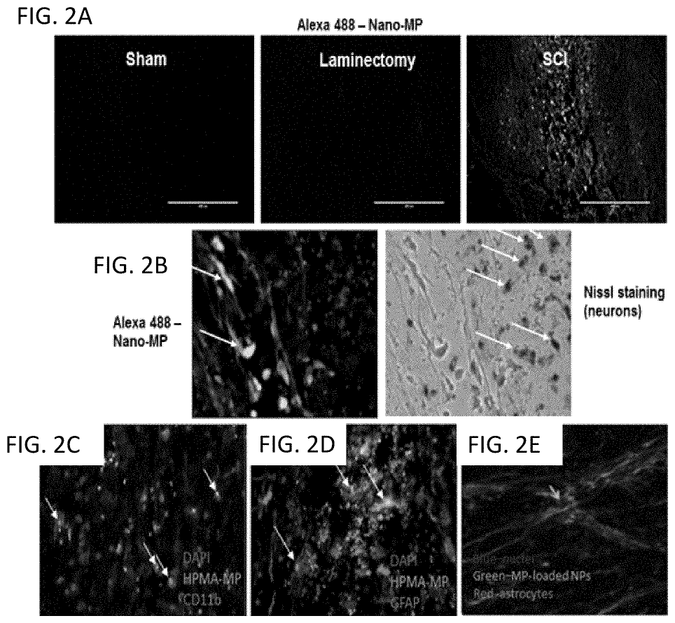

Microglia and astrocytes play a fundamental role in the secondary events following CNS injury. It was reported that dendrimer nanoparticle-based MP was internalized by glia cells [1]. To further evaluate the sub-localization of Nano-MP-Alexa, 10 μm longitudinal spinal cord sections were examined by fluorescent microscopy. Similarly, Nano-MP-Alexa was observed only in the spinal cord of injured animals (FIG. 2A). Alexa 488-Nano-MP was weakly detected in spinal cord motor neurons, as demonstrated by nissl (FIG. 2B) and SM32 staining (data not shown), but accumulated to a far greater extent in CD11+ microglia and GFAP⁺ astrocytes [FIG 2C-E]. It has been reported that internalized nanoparticles (CMCht/PAMAM-MP) are present at the lesion site of the spinal tissue 3 h after the injury. By comparison, the Nano-MP-Alexa 488 [2C, 2B] showed significantly higher fluorescent signal in spinal tissue at the injury site than CMCht/PAMAM-MP-FITC could achieve [2E]. These findings have demonstrated that a single administration of the Nano-MP is able to be preferentially delivered to the site of the injured spinal cord, where the pro-drug is sequestered and retained for several days, predominantly in microglia and astrocytes.

### ii. Nano-MP Attenuated SCI-Induced Oxidative Stress in The Spinal Cord

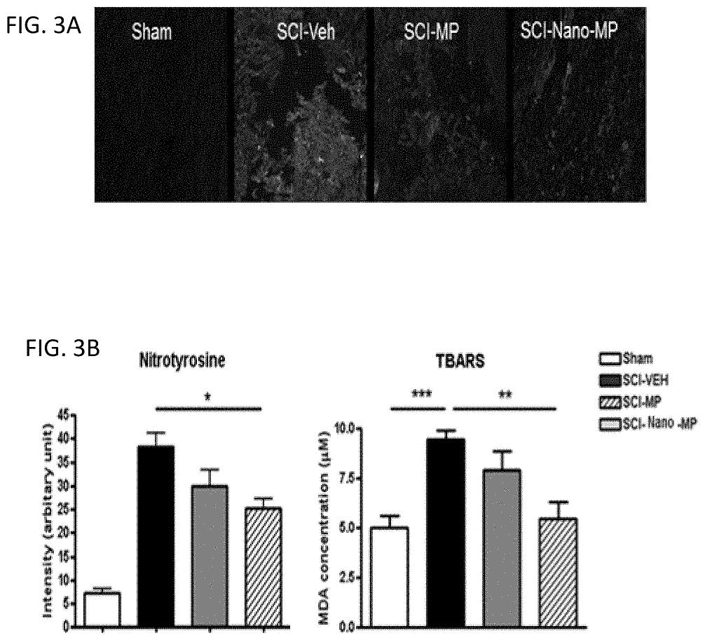

Oxidative stress is considered a hallmark of spinal cord injury. The glucorticoid steroid methylprednisolone has been reported to have significant antioxidant activities and improve the recovery of SCI patients in clinical trials mainly through the inhibition of reactive oxygen-induced lipid peroxidation and inflammation. To test whether MP-Nano administration, compared to that of free MP, will offer similar or superior ability to reduce lipid peroxidation or oxidation, inflammation and neural damage following SCI, we first measured the level of nitrotyrosine (NT), an indirect chemical indicator of toxic nitric oxide (NO) and peroxynitric-induced cellular damage, in the spinal cord at 2 and 7 days post transection. Both Nano-MP and MP significantly decreased SCI-induced accumulation of nitrotyrosine (−45.9% and −40.0% vs. SCI-veh, respectively) at 7 days post injury, whereas only Nano-MP was able to reduce the NT accumulation (−29.9% vs. SCI-veh) at 2 days post injury (FIG. 3A).

To determine the status of lipid peroxidation in injured spinal cord tissues, a TBARS assay was used to assess the levels of malondialdehyde (MDA) as an indication of lipid peroxidation. Acute SCI significantly increased the MDA level in the spinal cord as compared to sham control animals (9.437±0.4869 μM vs. 5.003±0.6249 μM, N=5-6). Notably, only Nano-MP treatment, not systemic MP treatment, decreased the MDA level (5.472±0.8502 μM, N=7), thus protecting the animals from SCI-mediated MDA accumulation and lipid peroxidation in the spinal cord (FIG. 3B).

### iii. Nano-MP Reduced Inflammation and Injury-Related Cellular Markers in the Spinal Cord

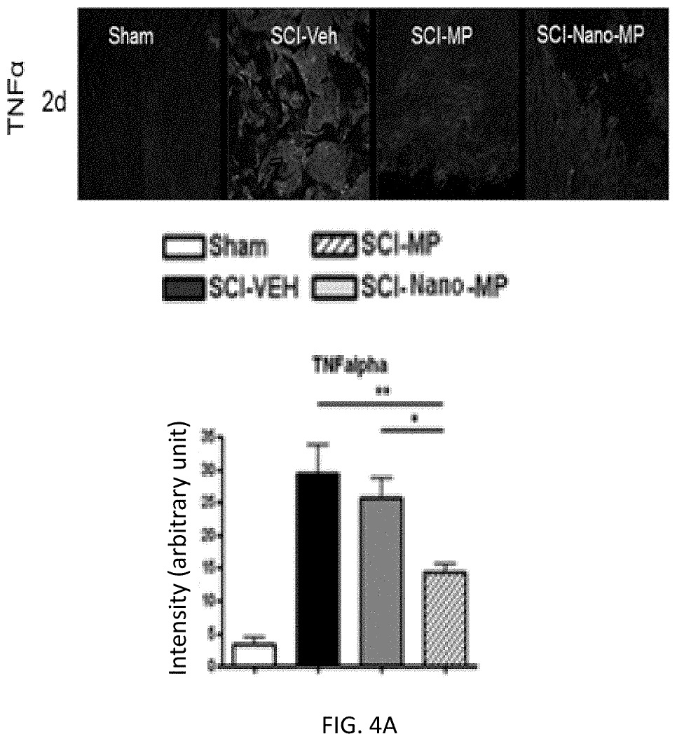

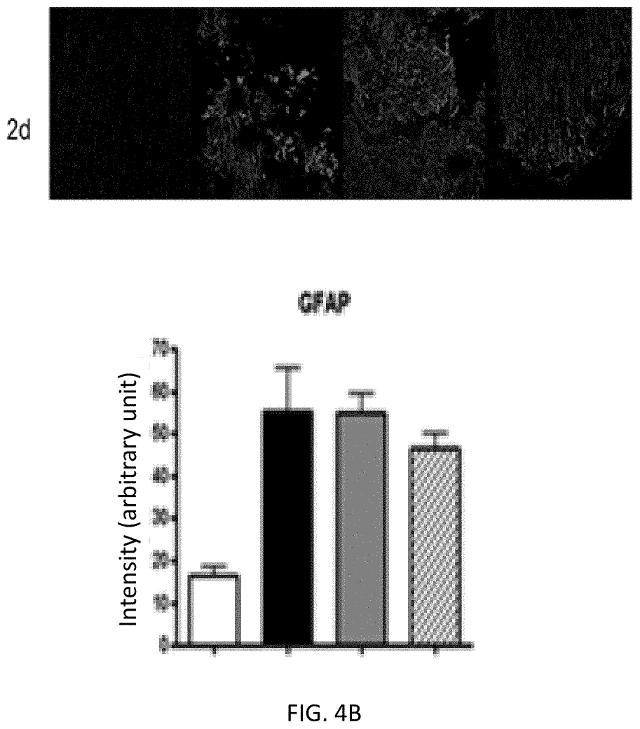

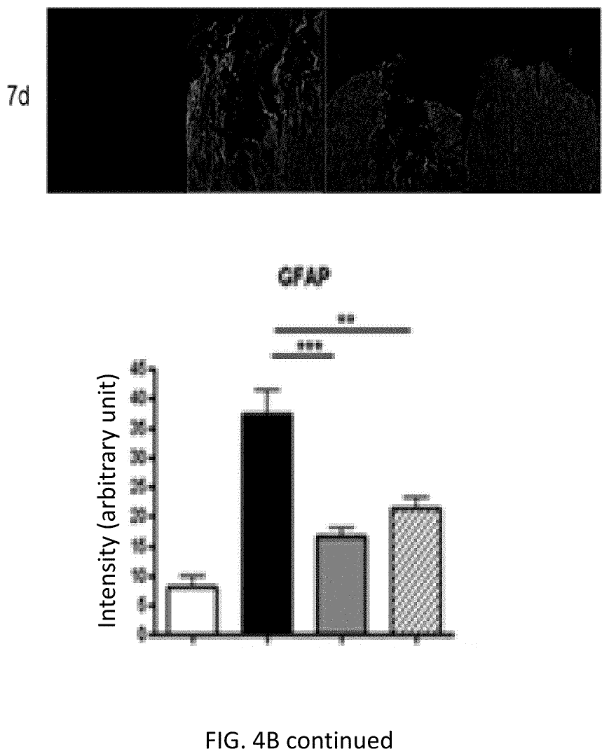

Inflammation responses are a major element of secondary injury and play a pivotal role in regulating the pathogenesis of acute and chronic SCI. From 3 to 24 hours post SCI, upregulation of TNF-α could be detected around the injured area. To determine whether Nano-MP could provide a better protection against inflammatory responses than systemic MP, the expression of TNF-α in the spinal cord at 2 and 7 days post injury was evaluated. As expected, acute SCI led to increased TNF-α reactivity in the spinal cord. At 7 days post injury, Nano-MP decreased SCI-induced TNF-α expression to the same level as systemic MP treatment (−33.9% and −32.5% vs. SCI-veh, respectively). Interestingly, similar to oxidative stress markers, TNF-α also showed a significant decrease (51.3% vs. SCI-veh) only upon Nano-MP, not MP treatment at 2 days after injury (FIG. 4A).

### iv. Nano-MP Administration Significantly Reduced Neural Damage and Enhanced Neuroprotection after Acute SCI

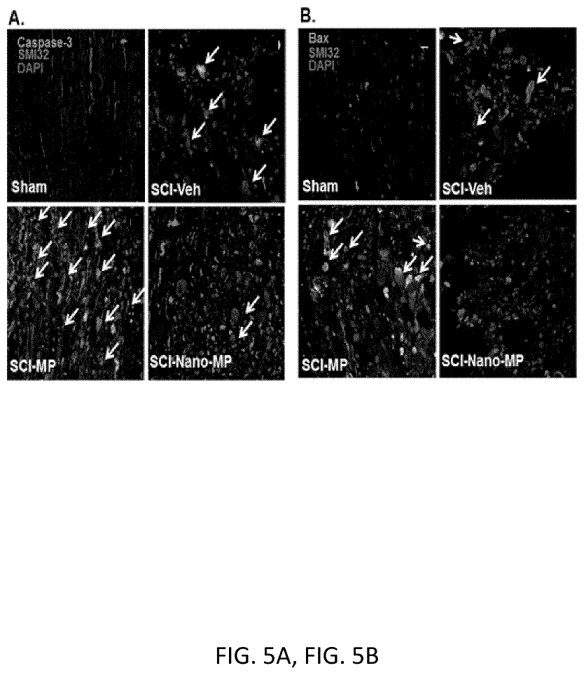

Apoptosis, as demonstrated by nuclear DNA fragmentation and caspase activation, was a prominent feature in the spinal cord post SCI. After SCI, some cells at the lesion site die by post-traumatic necrosis, whereas others die by apoptosis. Pro-apoptotic proteins (e.g., Bax) and caspase-3 activation plays a central role in cell apoptosis. As expected, acute SCI led to increased caspase-3 activation (green immunostaining; FIG. 5A) and Bax expression (green immunostaining; FIG. 5B). Further examination of cellular localization of caspase-3 and Bax in injured spinal cord tissue sections revealed that the caspase-3 and Bax immunoreactive signal selectively co-localized with motor neuron-specific immunoreactivity as determined by overlapping caspase-3/Bax and SM32 staining. The caspase-3 and Bax immunoreactive signal were significantly stronger in SCI-Veh animals than those in SCI-MP animals, indicating that MP administration causes further neural apoptosis after SCI. Importantly, Nano-MP locally delivered to injured spinal cord significantly reduced the immunoreactive signals of Caspase-3 activation and Bax in motor neurons, indicating that a single dose of the Nano-MP administration significantly reduced neural damage and enhanced neuroprotection in the spinal cord after acute SCI. This data suggest that targeted delivery of Nano-MP nanoparticles was able to more effectively protect animals from SCI-induced apoptosis, compared to systemic MP administration.

### 4. Discussion

Spinal cord injury (SCI) is a catastrophic medical problem that causes loss of sensory, motor, and autonomic function. To date, despite tremendous efforts made to the contrary, there are no fully restorative therapies for SCI. In both human and animal studies, systematic high-dose MP (30 mg/kg) delivery has shown beneficial effect on spinal cord injury. The use of systemic high-dose MP in acute SCI has been controversial due to its effect on functional recovery are modest and largely offset by a number of adverse effects including infection, myopathy, neuropathy, and disorders of carbohydrate metabolism. The side effects caused by systemic high-dose MP therapy can be reasonably related to high systemic dosage and associated toxicity.

Recently a local, sustained delivery of MP via dendrimer nanoparticles was developed and tested in a rat model of SCI. The study reported that relative to systemic delivery, local MP-nanoparticle therapy significantly reduced lesion volume and improved behavioral outcomes, indicating that local delivery nanoparticle-conjugated MP presents an effective method for introducing MP locally after SCI and significantly enhances therapeutic effectiveness compared to unmodified MP administered either systemically or locally. Similarly, a MP-loaded polycaprolactone based nanoparticle was developed and embedded in an implantable fibrin gel for topical delivery to injured spinal cord. This nanoparticle-gel system showed similar results with systemic high dose MP administration. However, both approaches involved a surgical procedure for implanting the encapsulated MP or embedded fibrin gel onto the lesion site, which may not be practical for many SCI patients.

A nanotechnology-based, polymeric delivery systems (a polymeric methylprednisolone conjugate that selectively delivers MP to the sites of injured and/or inflamed spinal cord, minimizing unwanted distribution and exposure to other tissues was developed. There are several potential advantages of this polymeric methylprednisolone conjugate-mediated approach: 1) Remarkable lower dose: Due to the fact that intravenous MP has a short pharmacokinetic half-life (2.5-3 h) and p-glycoprotein mediates exclusion of MP from the spinal cord, systemic administration of MP requires a high-dose MP regimen leading to high-dose-associated adverse effects. A significant lower dose of nanoparticle-conjugated MP was used that is equivalent to 20% of MP at 30 mg/kg. 2) More efficient, targeted delivery to the injury site: systemically administered nanoparticle-conjugated MP was selectively delivered to injured spinal cord, much more convenient than surgical procedures to locally deliver the drug to lesion sites. 3) Better therapeutic effect: targeted delivery enhanced the therapeutic effectiveness by increasing the local dose of MP at injury sites. The targeted delivery of the polymeric methylprednisolone conjugate demonstrates significantly improved therapeutic effects on the injured spinal cord when compared to systemic MP delivery. 4) Injectable and lyophilized powder formulation: the polymeric methylprednisolone conjugate can be stored as lyophilized powder and easily resuspended for injection purpose.

The data demonstrate that systemic administration of a polymeric methylprednisolone conjugate in a rat model of SCI preferentially extravasates through injured and “leaky” vasculature at sites of neurological injury to the spinal cord, where the drug is sequestered and retained by infiltrating inflammatory cells. Therefore, the local cells become the drug depot, and gradually release the active MP in response to the low pH microenvironment caused by an imbalance between enhanced metabolic activity and insufficient oxygen supply from inflamed cells. Selective delivery of the polymeric methylprednisolone conjugate to the sites of injury significantly decreased the reactivity to early markers of injury/secondary injury at an earlier time point (2 days post injury) than systemic MP administration (7 days post injury), indicating that this polymeric methylprednisolone conjugate ca serve as an innovative therapeutic agent for clinical applications in patients with SCI.

### B. Low Dose of Methylprednisolone Administration via Nanoparticles Prevented Glucocorticoid-Induced Muscle Atrophy and Osteoporosis in an Animal Model of Acute Spinal Cord injury

### 1. Introduction

Although immediate administration of methylprednisolone (MP) after spinal cord injury has been suggested to improve functional outcome, the safety of this approach has been questioned because of the well-recognized adverse effects of glucocorticoids on skeletal muscle, including muscle atrophy and glucocorticoid myopathy. Skeletal muscle atrophy is characterized by a decrease in the size of the muscle fibers. Glucocorticoids have been shown to cause atrophy of fast-twitch or type II muscle fibers (particularly IIx and IIb) with less or no impact observed in type I fibers. Exposure of myotubes or skeletal muscle to glucocorticoids increases the transcription factor FOXO gene expression, particularly -1 and -3a. Several genes (Atrogin-1, MuRF-1, Cathepsin-L, PDK4, p21, Gadd45, 4E-BP1) controlled by the FOXO transcription factors are strongly induced in microarray analyses of muscle atrophy due to a variety of wasting diseases. Administration of high-dose methylprednisolone for 24 h reduced muscle size and increased atrophy-related gene expression in acute SCI rats.

SCI patients have an obvious reduction in whole body glucose transport that seems proportional to their reduction in muscle mass. Retrospective analysis of acute SCI patients revealed the association of hyperglycemia with a worse functional outcome, irrespective of the subject's history of diabetes mellitus. Hyperglycemia due to increased gluconeogenesis and insulin resistance is another common side effect after glucocorticoid treatment. Development of a significant transient hyperglycemia was reported in acute spinal cord injury patients that received high-dose methylprednisolone treatment. Insulin resistance, a side effect of a more inactive lifestyle, seems to contribute to SCI-related osteoporosis.

Several distinctive features are associated with SCI-related bone loss, including permanent immobilization, neurological dysfunction, systemic hormonal alternations and associated metabolic disorders. High bone loss is detected mainly at the distal femur and proximal tibia, where fracture predominantly occurs. As a consequence of acute SCI, abnormal skeletal unloading dysregulates bone metabolism with a marked depression of osteoblastic bone formation as well as a profound increase in osteoclastic bone resorption. Glucocorticoid-induced osteoporosis is one of the most common and severe adverse effects related to glucocorticoid treatment. The effects of MP administration on SCI-related bone loss have recently been evaluated by our group. One day of MP at a dose comparable to those routinely employed in clinical practice immediately after SCI resulted in a worsened loss of bone mass and integrity below the level of lesion, associated with elevations in expression of genes involved in pathways associated with osteoclastic bone resorption.

MP therapy-related side effects are most likely due to the high systemic dosage and associated toxicity, and the relatively modest neurological gains are thought to be due to inadequate and inefficient dosing to the injury site. Therefore, targeted MP delivery to the injury site can reduce systemic side effects, enhance efficacy, and eventually improve neurological outcome and clinical care. Recent advances on drug delivery technology by nanomedicine provide the ideal platform for a targeted delivery of the drug to diseased tissues/cells. One such approach is the synthesis of polymer conjugates, in which drugs are covalently attached to the polymer carrier via a cleavable linker (e.g., N₂-hydroxypropyl methacrylamide (HPMA), polyethylene glycol (PEG)) that can be degraded in specific biological microenvironments. A novel water-soluble-HPMA-based acid-sensitive polymeric delivery system (a polymeric methylprednisolone conjugate) was developed to selectively deliver dexamethasone (Dex) to inflamed joints in adjuvant-induced arthritis (AA) rats. Since acute SCI results in extensive inflammation at injury sites, which is one of the same pathological features observed in AA, we speculate that nanoparticle-based local delivery successfully used in the treatment of AA in preclinical animal models might also be applicable in the conditions of acute SCI.

In this study, the effects of polymeric methylprednisolone conjugate delivery onto the injured spinal cord on glucose metabolism, muscle atrophy and bone loss were examined in an animal model of acute SCI. Results of these outcome measurements were quantified and compared with laminectomy only animals (Sham), vehicle-treated SCI animals (SCI-veh) and systemically administrated MP (30 mg/kg, single dose) animals (SCI-MP).

### 2. Material and Methods

### i. Animals Surgery, Drug Administration and Tissue Collection

All animals were maintained on a 12:12-h light/dark cycle with lights on at 07:00 h in a temperature-controlled (20±2° C.) vivarium. Spinal cord transection surgery was performed as previously described. In brief, 9-week-old Wistar rats (Charles River) were anesthetized by inhalation of isofluorane (3-5%) and hair was removed with a clipper. Skin over the back was cleaned with betadine and isopropyl alcohol. After making a midline incision the spinal cord at the site of transection (T4) was visualized by laminectomy, and the spinal cord was transected with microscissors. The space between transected ends of the spinal cord was filled with surgical sponge and the wound was closed in two layers with suture. Urine was manually expressed 3 times daily until automaticity develops, then as needed. Baytril was administered for the first 3 to 5 days postoperatively then as needed for sign of cloudy or bloody urine or wound infection. Sham-transected animals received a laminectomy-only surgery.

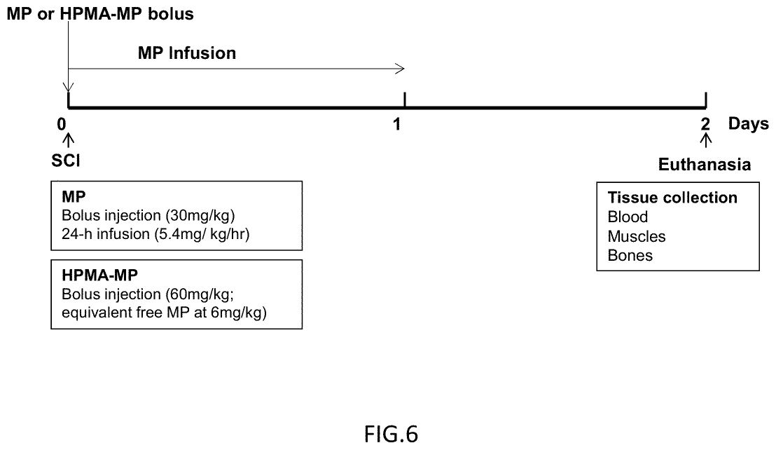

Injectable methylprednisolone (MP) solution (62.5 mg/ml) was obtained from Pfizer. MP was conjugated to the chain terminus of methoxy HPMA, an amphiphilic macromolecular prodrug to produce a polymeric methylprednisolone conjugate. Immediately after spinal cord transection, animals were administered an intravenous injection of freshly prepared MP-HPMA (60 mg/kg), MP (30 mg/kg) or vehicle (propylene glycol) by tail vein injection, followed by implantation of an Alzet pump (Durect Co, Cupertino, Calif., USA) into a subcutaneous pocket, which provided a 24-hr infusion of MP at 5.4 mg/kg/hr in SCI-MP group, or vehicle in SCI-veh and SCI-Nano-MP groups. These dosing of MP corresponds on an mg per kg basis to that prescribed by the Bracken protocol. The MP dose in SCI-Nano-MP animal group is equivalent to 3.75% of the free MP dose in SCI-MP animals. Sham-transected animals underwent laminectomy and implantation of the same Alzet pumps, with vehicle (propylene glycol) infusion instead, as baseline controls. The design of the two studies is shown in FIG. 6.

At the end of animal study, animals were euthanized by inhalation of isofluorane prior to harvesting of tissue for study. Gastrocnemius, soleus, plantaris, EDL, bicep and tricep muscle on both sides were dissected, weighed and stored at −80° C. for further analysis. The leg was removed using sterile technique; careful dissection was performed to free the head of the femur from the pelvis. To preserve bone for micro-CT study, the left leg was removed and placed into tubes containing 4% PFA overnight, after which the PFA will be replaced with 70% ethanol for storage. The right femur and tibia (n=4-5 per group) were placed in ice-cold Minimum Essential Alpha Medium (α-MEM), and then immediately processed for extraction of total RNA from bone.

### ii. Measurement of Blood Glucose Level

Rats were fasted overnight in clean cages with free access to water in new clean bottles. The next morning prior to sacrifice, fasted blood glucose measurement was taken by applying tail blood to a Contour Blood Glucose Monitoring System (Bayer).

### iii. Muscle Histology

Pieces of gastrocnemius muscle were frozen in isopentane pre-cooled on dry ice and stored at −80° C. Transverse sections (10 μm) were then cut on Leika CM3050S cryostat and mounted directly onto cooled glass slides followed by hematoxylin & eosin (H&E) staining. Stained sections were viewed in the EVOS FL Auto system (Life Technologies). Fiber size measurement and distribution was performed using the ImageJ software (NIH).

### iv. Muscle RNA Extraction and Western Blot

Total RNAs from gastrocnemius, soleus and bone were extracted using the TRI reagent (Sigma-Aldrich). For western blot analysis, gastronemius and soleus muscle tissue were lysed in RIPA buffer. Protein concentrations were determined using the BCA method. 30 μg protein lysate was used for the SDS-PAGE protein separation, and blots were probed with FOXO1 antibody.

### v. Dual-Energy X-Ray Absorptiometry and μCT

Areal bone mineral density (BMD) measurements were performed by using a small animal dual-energy X-ray absorptiometer (DXA) (Lunar Piximus, Fitchburg, Wis., USA) as previously described. Volumetric BMD and bone architecture of the distal femur were assessed by a Scanco μCT scanner (μCT-40; Scanco Medical AG, Bassersdorf, Switzerland) at 16 mm isotropic voxel size as previously described.

### vi. Extraction of Total RNA from Bone

Total bone RNA was extracted as previously described with some modifications [26]. Briefly, long bones were dissected free of soft tissues, and bone marrow were flushed away with PBS using a 27 G ½ needle-syringe. The bone samples (˜1 g) were longitudinally cut into small piece and then digested 3 times with 2 mg/ml collagenase type I (Gibco, >150 Units; 20 ml), one time with 5 mM EDTA (Sigma-Aldrich, 10 ml)), and one more with the collagenase and EDTA, each for 25 min on a Shaker with rotation at 150 rpm at 370 C. Following the digestions, the bone samples were crushed using a mortar and pestle in liquid nitrogen. RNA was extracted from the lysate using the TRizol reagent (Sigma Aldrich) according to the manufacturer's instructions.

### vii. Quantitative PCR

Real time PCR was used for the determination of mRNA levels as described previously. One μg of total RNA was used to synthesize the first strand cDNA by the High Capacity cDNA Reverse Transcription Kit (Applied Biosystems). qPCR was performed with an Applied Biosystems (ABI) Via 7 thermal cycler using ABI Taqman 2×PCR mix and ABI Assay on Demand qPCR primers. Changes in expression were calculated using the 2-ΔΔCt method using 18S RNA as the internal control.

### viii. Statistics

Data are expressed as mean±SE; the number of independent samples (n) is provided in the legend of each figure. The statistical significance of differences among means was tested using one-way analysis of variance and Bonferroni's post hoc test to determine the significance of differences between individual pairs of means using P<0.05 as cutoff. Statistical calculations were performed using Prism (GraphPad Software, La Jolla, Calif., USA).

### 3. Results

### i. Body Weight, Food Intake and Glucose Metabolism

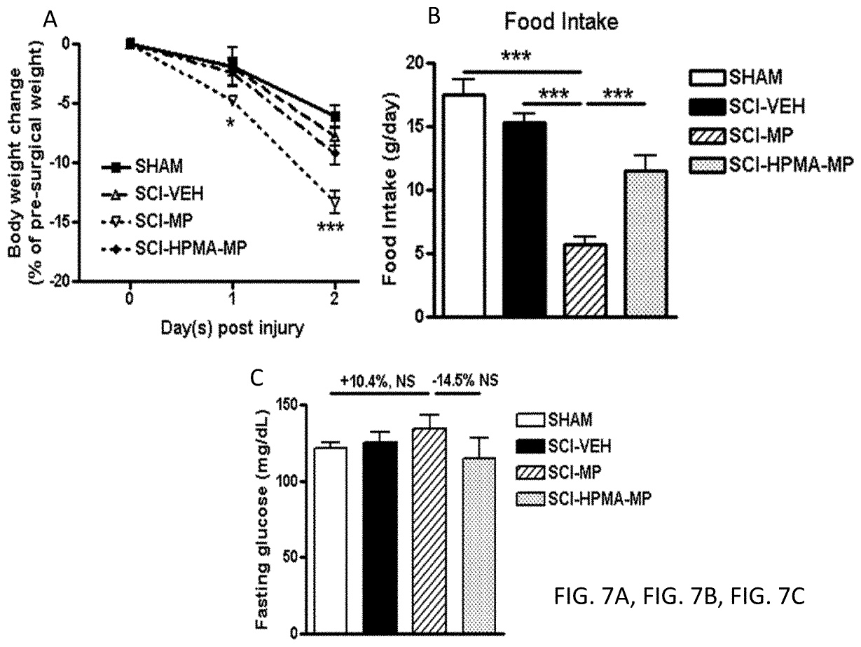

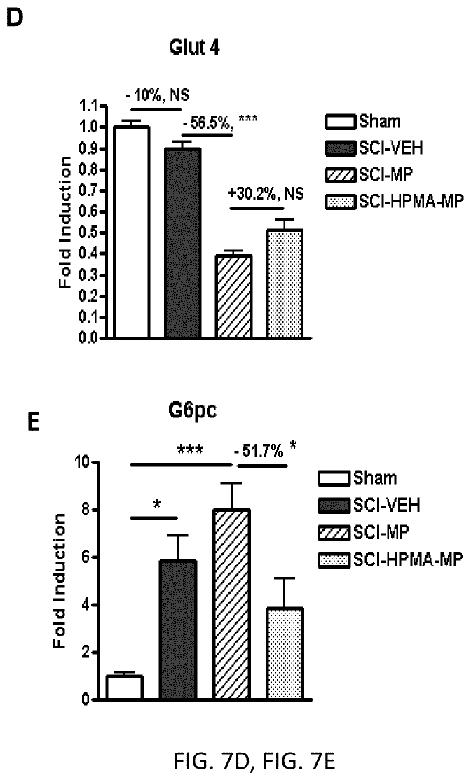

The observed body weight changes and food consumption after injury and MP or Nano-MP treatment are shown in FIG. 7A. Decrease of body weight is found in all experiment groups including the control animals that received sham operation (FIG. 7A), which may be attributed to the stress of surgery. Comparing to control animals, significantly higher weight loss is only detected in SCI animals receiving systemic MP injection at both Day 1 (p<0.05) and Day 2 (p<0.001) post injury (FIG. 7A). In contrast, no body weight loss was found in Nano-MP animals with no difference from those of SCI-vehicle or Sham control animals. Pronounced decrease of food intake was detected in SCI animals receiving systemic MP injection when compared to all other experiment groups (p<0.001, FIG. 7A), indicating that the combination of surgery and systemic MP treatment may result in more stress.

Glucocorticoids are the key regulators of both stress and energy balance, so the glucose level (FIG. 7B) was also measured at 2 days post injury. A marginal increase of fasting glucose level was detected in SCI-MP animals (+10.4% vs. sham) but it did not reach statistical significance. Considering that SCI-MP rats had a much lesser food intake which would lead to a lower glucose level in the blood, the actual impact of SCI-MP on the glucose metabolism would be higher than what we could conclude from the numbers. Notably, the glucose level in SCI-HPMA-MP rats was about −14.5% lower than SCI-MP rats, indicating that nanoparticle-conjugated MP injection did not have any negative effect on the fasting glucose level.

The gene expression level of GLUT4, an insulin-regulated glucose transporter found primarily in adipose tissues and striated muscle (skeletal and cardiac) and Glucose-6-phosphatase, catalytic subunit (G6PC), a key enzyme in glucose homeostasis that functions in gluconeogenesis and glycolysis were also examined. Systemic MP administration in SCI animals decreased GLUT4 expression by −56.5% (p<0.001, FIG. 7C). Although it did not reach statistical significance, the GLUT4 expression level in the SCI-HPMA-MP group, is about +30% higher than that in the SCI-MP group. SCI significantly increased G6PC level (˜6 fold, p<0.05, FIG. 7D) as compared to sham control animals. Systemic MP injection further induced (˜8 fold vs. sham, p<0.001) G6PC expression whereas nanoparticle-conjugated MP delivery in SCI animals resulted in a marked decrease of G6PC level (−51.7%, p<0.05) as compared to SCI-MP group.

### ii. Nano-MP Administration, Compared to that of Free MP, Reduces Adverse Effects on Muscle after Acute SCI

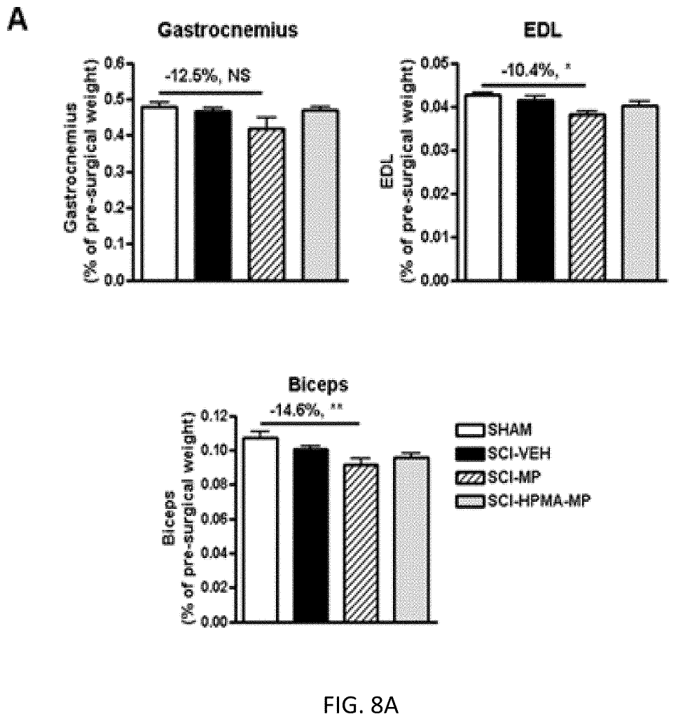

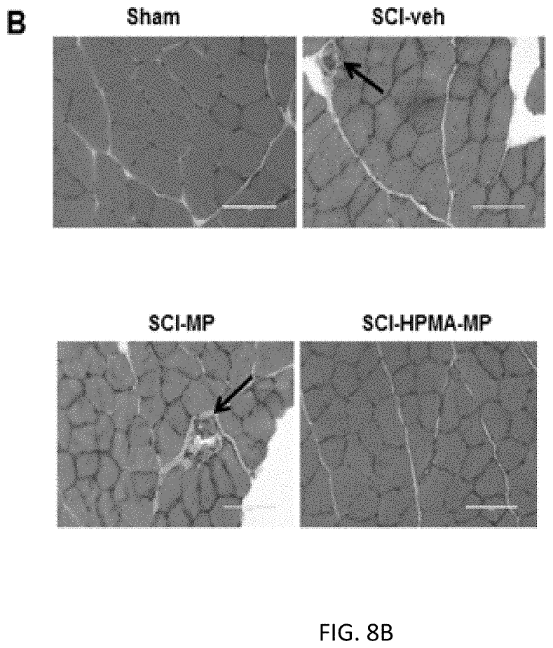

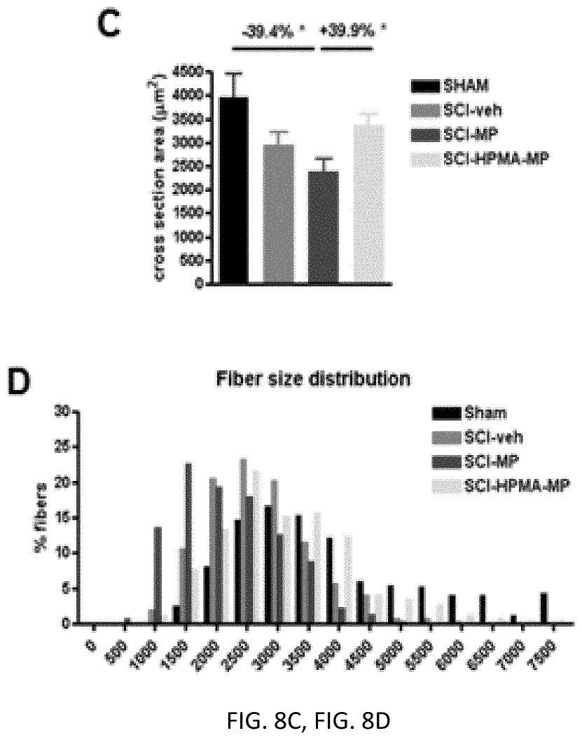

Muscle wasting is one of the major adverse effects of steroid therapy and substantial muscle waste could be the major contributor of body weight reduction. Comparing to sham-transected rats, reduction of muscle mass was detected in the EDL (−10.4% vs. sham, p<0.05), biceps (−14.6% vs. sham, p<0.01) and gastrocnemius (−12.5% vs. sham, NS) muscle of SCI-MP rats (FIG. 8A). The muscle mass of SCI-veh rats and SCI-HPMA-MP rats remained unchanged. Similar results were also found in the soleus, plantaris and triceps muscles (data not shown).

Examination of hematoxylin-eosin-stained sections of gastrocnemius muscle from SCI-Sham, SCI-veh, SCI-MP and SCI-Nano-MP animals was performed to evaluate the effects of MP or nanoparticle-conjugated MP on muscle structure. Necrotic fibers or fibers invaded by inflammatory cells were observed (FIG. 8B) in both SCI-veh and SCI-MP rats but not in SCI-Nano-MP rats. No fiber with centralized nuclei was found in any of the experimental groups, which is most likely due to the relatively short time period following injury. Unlike SCI-veh animals, which only showed marginal decrease in cross-sectional area and a minor left shift in fiber size, SCI-MP treatment dramatically reduced cross-sectional area by −40% (p<0.05, FIG. 8C) and resulted in a significant left shift of fiber size (FIG. 8D). Notably, SCI-HPMA-MP animals showed comparable cross-sectional area and similar fiber size distribution to control sham-operated animals.

### iii. Nano-MP Ameliorated Free MP-Induced Expression of Muscle Atrophy Markers

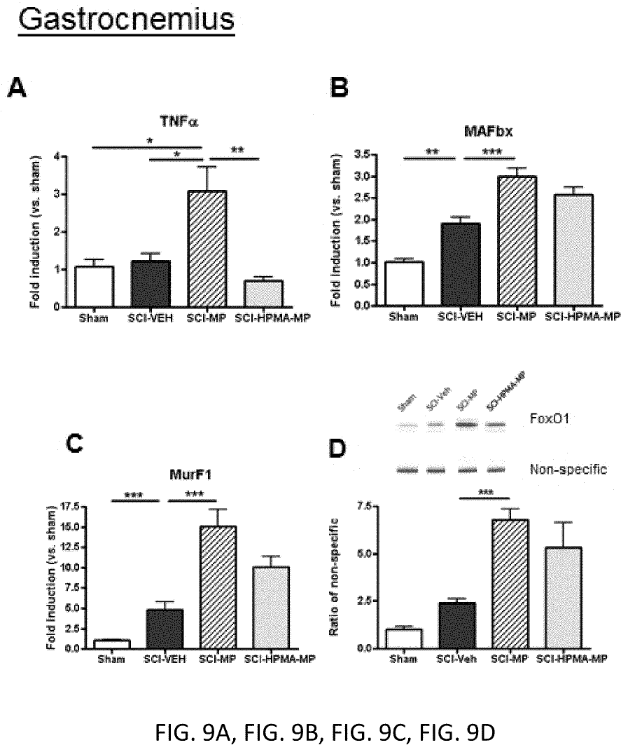

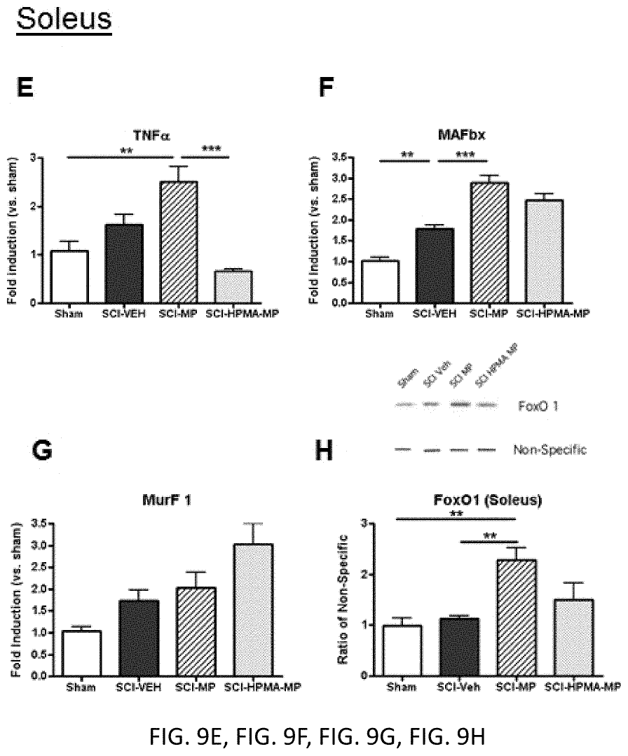

At 2 days post injury, systemic MP injection significantly increased the mRNA level of inflammatory marker TNF-α in the gastrocnemius (p<0.05, FIG. 9A) and soleus (p<0.01, FIG. 9E) muscle. In contrast, Nano-MP treatment did not change the expression level of TNFα as compared to sham-transected animals, suggesting that nanoparticle-conjugated MP treatment protected against SCI-induced inflammatory responses. The expression level of two muscle-specific E3 ubiquitin ligases, MAFbx/atrogin-1 and Muscle RING Finger-1 (MuRF1) were also examined. SCI increased the MAFbx (p<0.01, FIG. 9B) and MuRF1 (p<0.001, FIG. 9C) mRNA level in the gastrocnemius muscle, systemic MP injection in SCI animals (SCI-MP) resulted in a more significant induction of MAFbx and MuRF1 mRNAs. Slight decrease of MAFbx and MuRF1 mRNA level was found in SCI-HPMA-MP rats as compared to SCI-MP rats, but it did not reach statistical significance. A similar effect of MP or HPMA-MP on gene expression was observed for the soleus, with more significant increases of MAFbx (p<0.01, FIG. 9F) mRNA level in the SCI-MP group as compared to the SCI-veh group. FoxO1 is a key factor of muscle energy homeostasis through the control of glycolytic/lipolytic flux and mitochondrial metabolism. Comparing to SCI-veh group, systemic administration of MP drastically elevated FOXO1 protein level in both the gastrocnemius (p<0.001, FIG. 9D) and soleus muscle (p<0.01, FIG. 9H), whereas no significant increase of FOXO1 expression was detected in SCI-Nano-MP group.

### iv. Nano-MP Administration, Compared to that of Free MP, Reduces Adverse Effects on Bone after Acute SCI

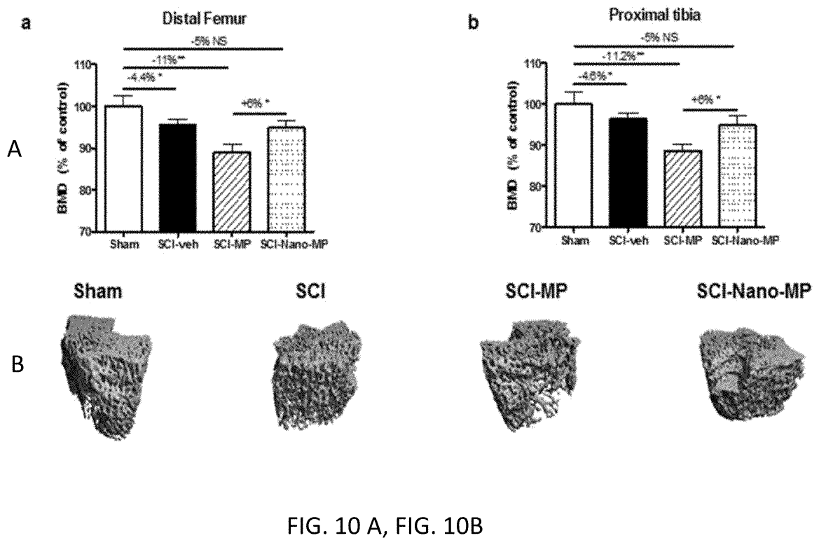

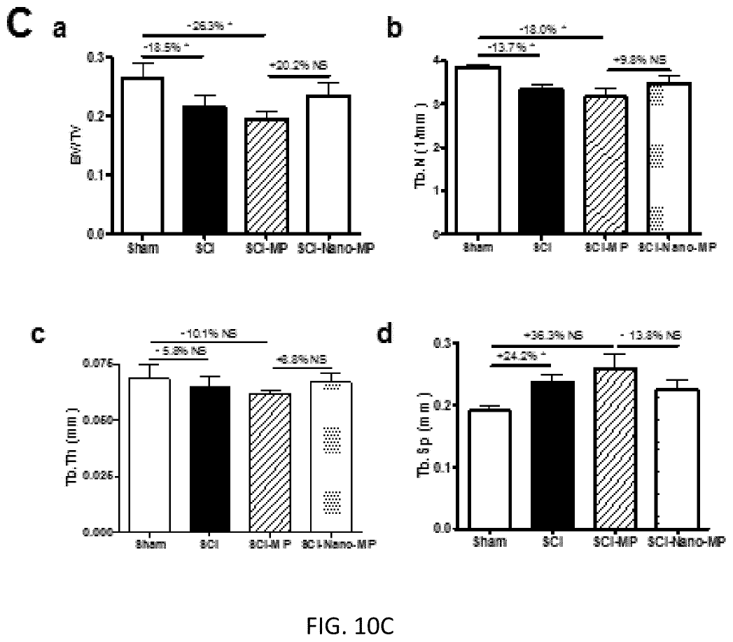

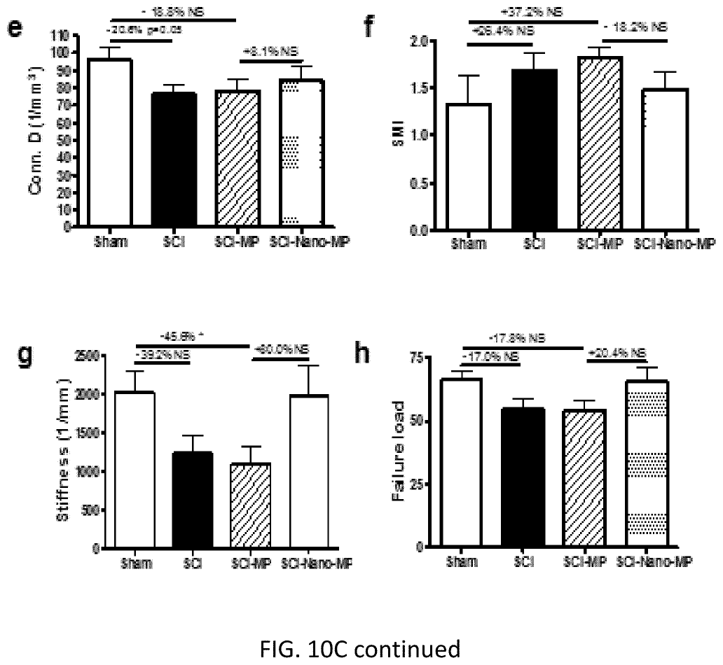

One of the inevitable complications of SCI is the associated osteoporosis. Using a small animal dual-energy X-ray absorptiometer (DXA), whether systemic MP injection or nano-conjugated MP injection could alter the bone loss in rats that received a complete spinal cord transection was examined. At the distal femur (FIG. 10A-a), SCI-MP rats showed an 11% decrease in the bone mineral density (BMD) as compared to sham control animals. SCI-HPMA-MP rats increased the BMD by 6% when compared to SCI-MP group. An almost identical BMD pattern was detected at the proximal tibia (FIG. 10A-b), with an 11.2% decrease of BMD in SCI-MP rats comparing with sham control animals. As reported previously, compared to Sham rats, a detectable yet not significant decrease in BMD was detected at the distal femur (—3.5%, FIG. 10A-a) and at the proximal tibia (−3.6%, FIG. 10A-b), despite of conducting the analysis at the relatively short period of time post injury.

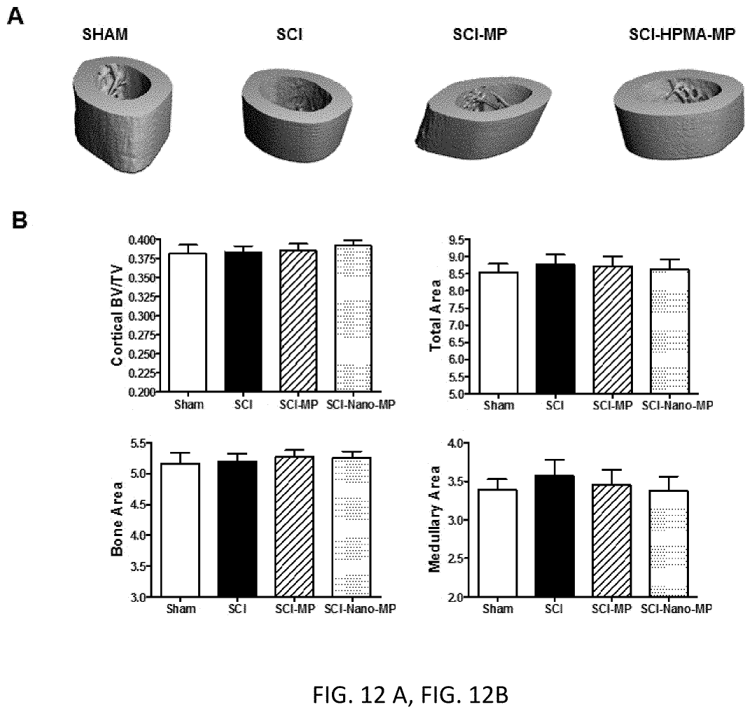

Bone architecture was then examined by high-resolution μCT to assess the changes in trabecular bone volume of the distal femur (FIG. 10B). As reported previously, SCI changed trabecular bone volume (−18.5%, FIG. 10C-a), trabecular bone number (−13.7%, FIG. 10C-b), thickness (−5.8%, FIG. 10C-c), separation (+24.2%, FIG. 10C-d), connectivity density (−20.6%, FIG. 10C-e), structure model index (+26.4%, FIG. 10C-f), stiffness (−39.2%, FIG. 10C-g) and failure load (FIG. 10C-h) as compared to sham control animals. Systemic MP administration in SCI animals resulted in a more significant change in each of those structural parameters than SCI-veh group (FIG. 10C). In contrast, MP delivery through nanoparticles in SCI animals was able to reserve bone architecture to a similar level as sham control animals (FIG. 10C). High-resolution μCT was used to assess the effect of systemic MP injection or nano-conjugated MP injection on the cortical architecture of the femur midshaft (FIG. 12). No significant change of cortical bone volume, cortical bone number, thickness or separation was observed.

### v. Nano-MP Alleviated Free MP-Induced Bone Resorption in SCI Animals

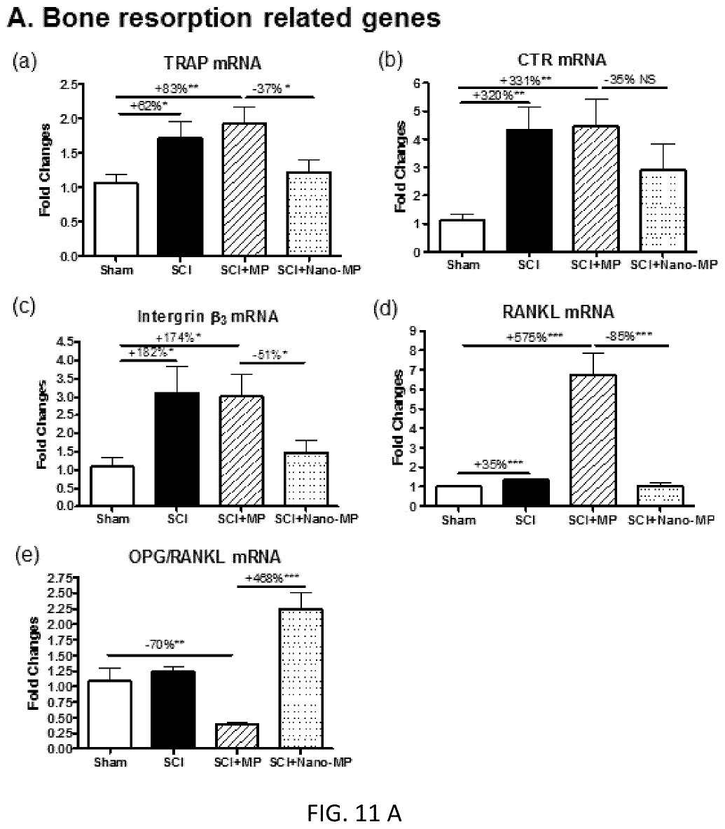

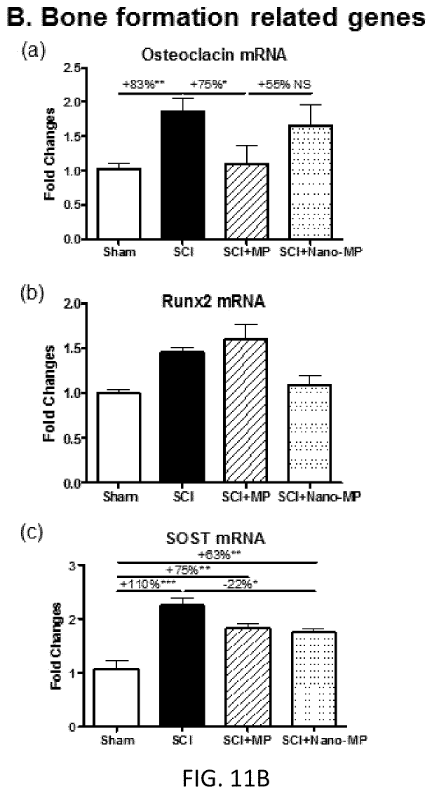

To examine the impact of MP or HPMA-MP on the expression of bone formation and bone resorption markers, we extracted whole bone RNA and performed quantitative PCR analysis. Comparing to sham control animals, increase of the osteoclastic markers TRAP (FIG. 11A-a), calcitonin receptor (CTR) (FIG. 11A-b) and Integrin β3 (FIG. 11A-c) mRNA was found in SCI-veh and SCI-MP groups, and to a lesser extent, in the SCI-HPMA-MP group. Remarkably elevated (˜7 fold) mRNA level of RANKL (FIG. 11A-d) was detected in SCI-MP group when compared with sham-transected animals, resulting in an about three-fold decrease in the OPG/RANKL ratio (FIG. 11A-e). However, expression of RANKL mRNA was significantly lower in SCI-HPMA-MP rats compared to that in SCI-MP group, a degree comparable to that observed in Sham animals; in particular, the OPG/RANKL ratio in SCI-HPMA-MP rats was increased by +468% when compared with SCI-MP group (p<0.001, FIG. 11A-e). Although slight increase of osteoblastic markers osteocalcin (FIG. 11B-a) and Runx2 (FIG. 11Bb) was also detected in SCI-veh and/or SCI-MP group, a comparison of the magnitude of gene expression suggested that bone resorption outweighed bone formation upon systemic MP injection after SCI. Importantly, compared to Sham animals, levels of SOST mRNA was significantly reduced by −22% (p<0.05, FIG. 11B-c) in SCI-MP group, which is otherwise elevated by +110% (p<0.001) after acute SCI.

### 4. Discussion

Although systemic delivery of high-dose MP has been shown to improve neurological outcomes after SCI, the use of high-dose MP in acute SCI has become controversial due to the accompanied severe side effect. Recently local, sustained delivery of MP via nanoparticles were developed and tested in rat model of SCI. The studies reported that relative to systemic delivery, local MP-nanoparticle therapy significantly reduced lesion volume and improved behavioral outcomes, suggesting that local delivery nanoparticle-conjugated MP presents an effective method for introducing MP after SCI and significantly enhances therapeutic effectiveness compared to unmodified MP administered either systemically or locally. However, both approaches utilized local delivery via surgical procedures, which may not be practical for many SCI patients. The impact of locally delivered MP on SCI-related muscle atrophy or bone loss has not been reported.

The aim of this study is to achieve high local dose of MP at trauma site via nanoparticles, and compare the pharmacological modulation of secondary damages including glucose metabolism, muscle atrophy and bone morphology and integrity between nanoparticle-conjugated MP and systemic administrated MP in acute SCI animals. A nanotechnology-based, polymeric delivery system (HPMA-conjugated macromolecule) has been developed that selectively delivers MP to the sites of injured and/or inflamed spinal cord through systemic injection to minimize unwanted distribution and exposure to other tissues.

Hyperglycemia has been reported in acute SCI patients receiving high dose MP treatment, which is consistent with the data that systemic MP administration increased blood glucose level in acute SCI animals along with significant decrease of glucose transport and increase of gluconeogenesis. Notably, acute SCI animals treated with HPMA-MP does not have severe glucose dysregulation as animals treated with high dose MP.

Glucocorticoids treatment is known to have adverse effects on skeletal muscle, including muscle atrophy and glucocorticoid myopathy. Administration of high-dose methylprednisolone for 24 h reduced muscle size and increased atrophy-related gene expression in rats received complete spinal cord transection at T10. In this study, a complete spinal cord transection at T4 was performed and significant reduction of muscle size and increased expression of atrophy markers in SCI animals treated with high dose MP was observed. SCI animals received HPMA-MP injection are resistant to high dose MP-induced muscle atrophy.

Glucocorticoids-induced osteoporosis is one of the most common and severe negative effect of glucocorticoid use. Glucorticoids decrease the differentiation and maturation of osteoblasts, leading to decreased bone formation and increased bone resorption. In addition to the use of glucocorticoids themselves, many of the diseases that they are used to treat, such as rheumatoid arthritis (RA), are associated with bone loss that is independent of glucocorticoid use. Bone loss following motor complete spinal cord injury is unique for its rate, distribution and resistance to currently available treatments. It is unknown how these complex pathophysiological changes are linked to molecular alterations that could influence bone formation and bone resorption. Proinflammatory cytokines involved in these pathophysical conditions impact bone formation and bone resorption. For example, in RA, TNF-α, interleukin (IL)-1, IL-6 and IL-11 increase RANKL expression, leading to an increased bone resorption. In consistence, elevated TNF-α expression as well as increased RANKL expression was found in the bone of SCI animals treated with high dose MP. There has been debate about the use of low-dose glucocorticoids in protecting bone loss in RA. In this study, nanoparticle-conjugated low-dose MP treatment prevented glucocorticoid-induced osteoporosis in acute SCI animals.

In summary, the data clearly demonstrate that systemic administration of a macromolecular MP prodrug in a rat model of SCI protects animals from high dose MP-induced glucose dysregulation, muscle atrophy and osteoporosis, suggesting that this nanoparticle-conjugated MP prodrug might serve as an innovative therapeutic agent with improved safety profile for clinical applications in patients with SCI.

Those skilled in the art will recognize, or be able to ascertain using no more than routine experimentation, many equivalents to the specific embodiments of the method and compositions described herein. Such equivalents are intended to be encompassed by the following claims.

1. A method of treating a spinal cord injury in a subject comprising: systemically delivering a composition comprising a polymeric methylprednisolone conjugate to the subject.

2. (canceled)

3. The method of claim 1, wherein the spinal cord injury is an acute spinal cord injury.

4. A method of inhibiting oxidative stress in the spinal cord of a subject comprising systemically delivering a composition comprising a polymeric methylprednisolone conjugate to the subject.

5. A method of inhibiting lipid peroxidation in the spinal cord of a subject comprising systemically delivering a composition comprising a polymeric methylprednisolone conjugate to the subject.

6. The method of claim 1, wherein the composition is administered intravenously.

7. (canceled)

8. The method of claim 1, wherein inflammation in the subject is reduced in the spinal cord.

9. The method of claim 1, wherein injury-related cellular markers are reduced in the spinal cord of the subject.

10. The method of claim 1, wherein motor neuron apoptosis is decreased in the spinal cord of the subject.

11. The method of claim 1, wherein only a single dose of the composition is delivered to the subject

12. (canceled)

13. The method of claim 1, wherein muscle and bone mass are not reduced in the subject.

14. The method of claim 1, further comprising administering a pain management therapeutic or anti-inflammatory agent to the subject.

15. The method of claim 14, wherein the administering of the pain management therapeutic or anti-inflammatory agent is co-administered with the composition comprising the polymeric methylprednisolone conjugate.

16. The method of claim 1, wherein the polymeric methylprednisolone conjugate comprises a polymeric carrier and one or more molecules of methylprednisolone.

17. The method of claim 16, wherein the polymeric carrier is a neutral water-soluble polymer.

18. The method of claim 17, wherein the neutral water-soluble polymer is N2-hydroxypropyl methacrylamide (HPMA) or methoxy HPMA.

19. (canceled)

20. The method of claim 16, wherein the polymeric carrier is conjugated to one or more molecules of methylprednisolone via a linker.

21. The method of claim 20, wherein the linker is a cleavable linker.

22. The method of claim 21, wherein the cleavable linker is an ester, hydrazone, acetal, ether, thiol ether, or amide linker bond.

23. (canceled)

24. (canceled)

25. (Canceled)

26. (Canceled)

27. The method of claim 1, wherein the polymeric methylprednisolone conjugate is present at a range between 1 mg/kg and 1000 mg/kg.

28. The method of claim 1, wherein the polymeric methylprednisolone conjugate is present at 60 mg/kg.
